(19) United States
(12) Plant Patent Application Publication
(10) Pub. No.: US 2011/0321205 P1
(43) Pub. Date: Dec. 29, 2011

(54) Black gum tree named "PRP1"

(50) ;

(76) Inventor: Phillip Powell, (US)

(21) Appl. No.: 12/803,464

(22) Filed: Jun. 28, 2010

(51) Int. Cl. A01H 5/00

(52) U.S. Cl. PLT/216

(57) ABSTRACT

A Black Gum named ‘PRP I’ having a well-branched, upright growth habit with a strong central leader, shiny dark green leaves in summer, and a crimson red fall leaf color and also capable of being asexually reproduced reliably from vegetative propagation.

### CROSS-REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates to a new and distinct variety of Nyssa sylvatica, Black Gum, referred to by its varietal named ‘PRP1’.

Discovery

I discovered ‘PRP1’ in October, 2003 growing in a cultivated landscape area of a home in Murray, Calloway County, Ky.

Propagation

‘PRP1’ was asexually propagated by side veneer grafting by the inventor in January, 2006. This propagation and the resulting progeny have proven the characteristics of my new variety to be firmly fixed. Further, subsequent observations have confirmed that my Black Gum tree ‘PRP1’ represents a new and improved variety of Black Gum tree as particularly evidenced by its well-branched, upright growth habit with a strong central leader, shiny dark green leaves in summer, and crimson red fall color, all of which can be reliably asexually reproduced.

Uniqueness

My tree ‘PRP1’ was observed to have a well-branched, upright growth habit with a strong central leader, shiny dark green leaves in summer, and crimson red leaves in the fall. These characteristics distinguish my tree ‘PRP1’ from other typical seedling Black Gum trees and known cultivars.

Use

My tree ‘PRP1’ was observed for a period of time in landscape and nursery settings, and is believed to be particularly useful as a specimen tree in commercial and residential areas, a street tree in residential areas. and in naturalized areas. It will also be useful to growers by enabling them to produce a higher quality tree with fewer inputs due to its upright growth habit and development of a central leader.

### BRIEF SUMMARY OF THE INVENTION

Black Gum trees are typically highly variable. The species Nyssa sylvatica is represented by two varieties, N. sylvatica var. sylvatica and var. biflora. In the fifth edition of the Manual of Woody Landscape Plants, 1998, Dr. Michael A. Dirr describes N. sylvatica var. biflora as being a smaller tree with smaller leaves when compared to N. sylvatica, but the main difference between these two varieties is habitat. Silvics of North America: 1. Conifers; 2. Hardwoods. Agriculture Handbook 654 describes the differences in more detail. Habitat is similar for both varieties. In areas where the two varieties overlap, it is difficult to distinguish between them. In the nursery trade, no effort is made to differentiate between the varieties due not only to their similarities, but also to the wide variation exhibited within the species as a whole.

Seedling Black Gum trees have a pyramidal shaped canopy in youth, and are usually well-branched, with many of the branches being pendulous. As the trees mature, the spreading and often horizontal branches will form an upright-oval, irregularly rounded canopy, or a flat-topped crown shaped canopy. Mature native Black Gum trees are typically 30-40 feet in height with a spread of 20-30 feet; although, some native specimens can reach 100 feet in height. In nursery settings, Black Gum trees often require a significant amount of effort to maintain a central leader. My Black Gum tree ‘PRP1’ is an improvement over the species in that it has a well-branched, upright growth habit with a strong central leader, shiny dark green leaves in summer, and crimson red leaves in the fall.

Black Gum trees are native from Maine, southern Ontario, and central Michigan to southern Florida and east Texas. N. sylvatica var. sylvatica grows in a variety of sites from the creek bottoms of the southern coastal plains to 3,000 foot high ridges in North Carolina. This variety grows best in well-drained, light-textured soils on low ridges of second bottoms and on high flats of silty alluvium; preferring lower slopes and terraces in the Southeastern United States. In upland areas it grows best in the loams and clay loams of lower slopes and coves. N. sylvatica var. biflora grows chiefly in the Coastal Plains in a variety of wet bottom land soils where it often intermingles with var. sylvatica.

Industry Representation

Cultivated Black Gum trees are represented in the industry by seedling material and a few cultivars. Seedling material is currently the industry standard, but many growers are starting to plant cultivars as they become more available. Seedling material is highly variable in growth rate, habit, and leaf characteristics, and overall consistency is difficult to maintain in a production system. Varietal differences within the species are as described above.

The Manual of Woody Landscape Plants lists seven cultivars. Of these, only Nyssa sylvatica ‘NXSXF’ U.S. Plant Pat. No. 11,391 Forum™ (hereinafter Forum™) and Nyssa sylvatica ‘Miss Scarlet’ (hereinafter ‘Miss Scarlet’) are described as having a red fall color. There is no mention of the growth habit of ‘Miss Scarlet’, but Forum™ is described as having an “upright, conical growth habit, uniformly spaced ascending branches, and consistent, red fall color” similar to my new selection. From the images in U.S. Plant Pat. No. 11,391, it is evident that the branching of my Black Gum tree ‘PRP1’ is more upright as compared to that of Forum™. The branches on Forum™ tend to emerge at a horizontal angle and then sweep upwardly, but return to a more horizontal angle as the tree ages. FIG. 4 of this application depicts the acute angle from which branches emerge from the main trunk of my tree ‘PRP1’. Based on the color terminology used in the Forum™ patent description and visual observations, my tree ‘PRP1’ also has a darker red fall color than the Forum™ tree. This is as shown in FIG. 1 which depicts the color of ‘PRP1’. Growers who have experience with the Forum™ tree suggest that leaf spotting could be a problem. However, my tree ‘PRP1’ has shown itself to be free from disease both in landscape and production settings, and the age and size of the parent tree are an indicator of its durability in a landscape.

Two new cultivars of Black Gum trees are becoming popular in the industry. Nyssa sylvatica ‘Wildfire’ (hereinafter ‘Wildfire’) and Nyssa sylvatica ‘Haymanred’ Red Rage™ (hereinafter Red Rage™) are both now being widely grown. ‘Wildfire’ was selected for the color of its new leaves, which emerge red and change with age to green. It is a strong grower with a central leader, but it has more horizontal branching than my new selection and is described as a broad grower. In contrast, my tree ‘PRP1’ is taller rather than wide and has a more upright growth habit. Red Rage™ was also selected for its fall color, which is orange-red. It is also a strong grower with a central leader, but it has much larger, longer leaves and horizontal branching which are significantly different for my Black Gum tree ‘PRP1’. FIG. 2 of this application provides a comparison of the fall color of leaves from my tree ‘PRP1’, and the Red Rage™ tree.

### BRIEF DESCRIPTION OF THE SEVERAL VIEWS OF THE DRAWINGS

The accompanying photographs depict the color of the tree and foliage of my new variety as nearly as is reasonably possible to make the same in a color illustration of this character, and other characteristics of my tree.

FIG. 1 depicts the fall color of my Black Gum tree ‘PRP1’. The dominant central leader can be clearly seen through the canopy of the tree.

FIG. 2 provides a fall color comparison between ‘PRP1’ and Red Rage™. The two leaves on the left side of the Figure are from my tree ‘PRP1’.

FIG. 3 depicts the summer leaf color of my tree ‘PRP1’.

FIG. 4 provides a comparison between a typical seedling Black Gum tree and my tree ‘PRP1’. ‘PRP1’ is shown on the left.

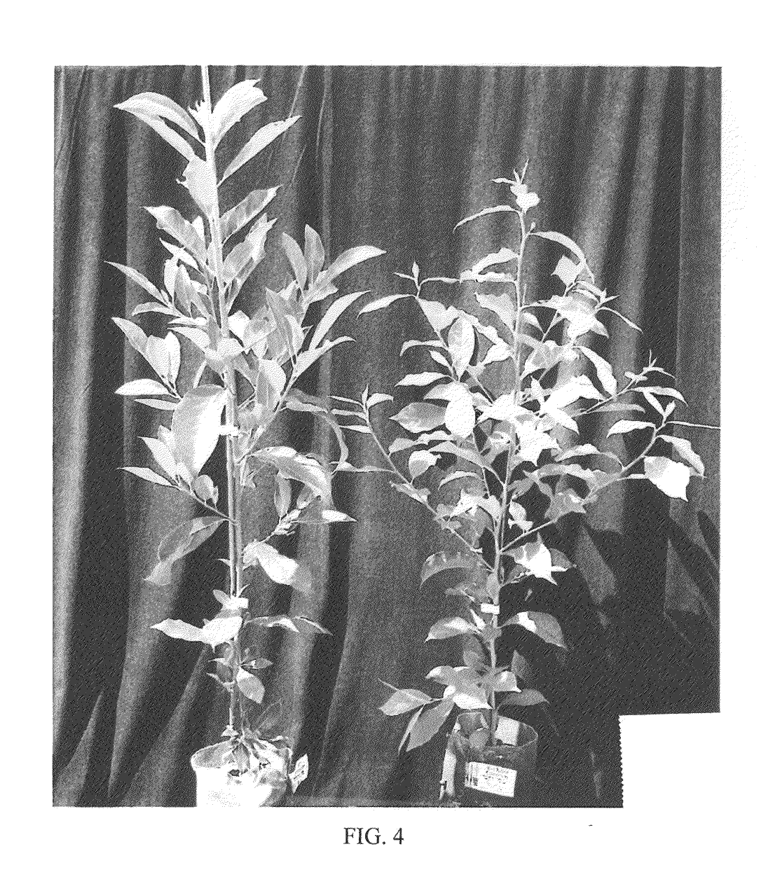

FIG. 5 provides a comparison between Red Rage™, ‘PRP1’, and ‘Wildfire’ trees. My tree ‘PRP1’ is in the middle.

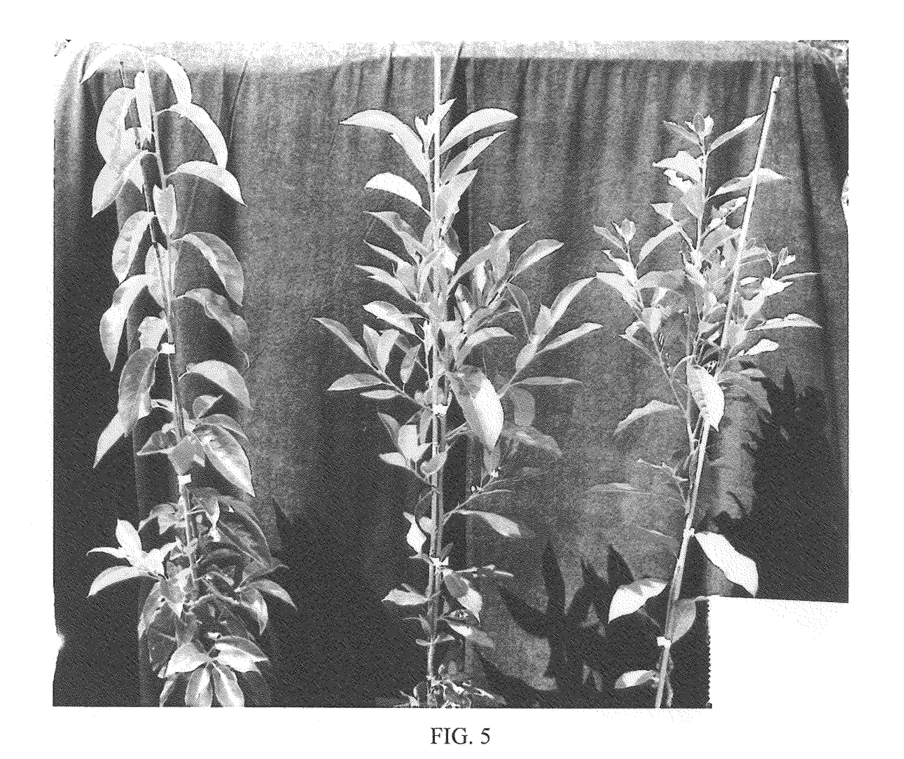

FIG. 6 depicts the strong central leader and upright branching habit of my tree ‘PRP1’.

FIG. 7 depicts the bark of the trunk of ‘PRPI’.

### DESCRIPTION OF THE PREFERRED EMBODIMENT

The following is a detailed description of my new variety of Black Gum with color terminology in accordance with The Royal Horticulture Society (R.H.S.) color chart. My Black Gum tree ‘PRP1’ has not been observed under all growing conditions and variations may occur as a result of different growing conditions. However, all progeny of my new variety of Black Gum tree, insofar as have been observed, have been identical in all the characteristics described below.

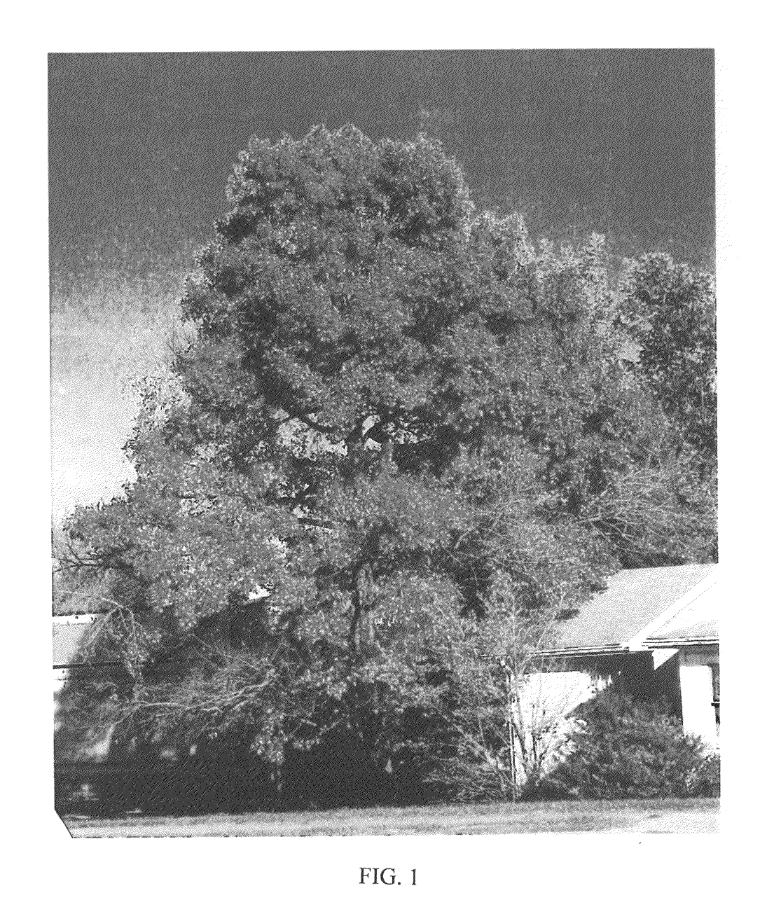

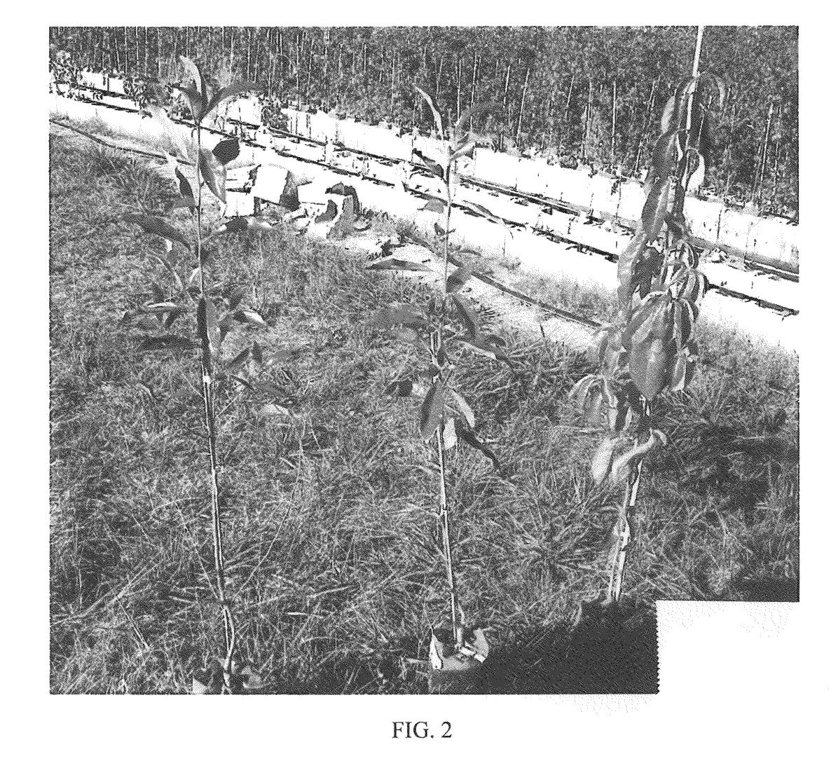

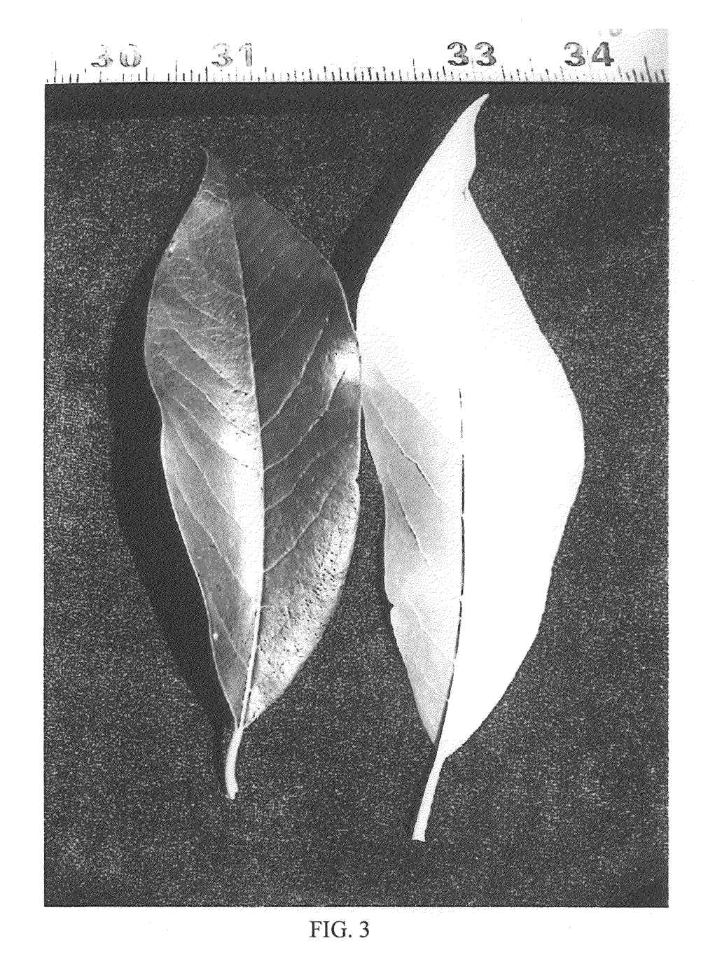

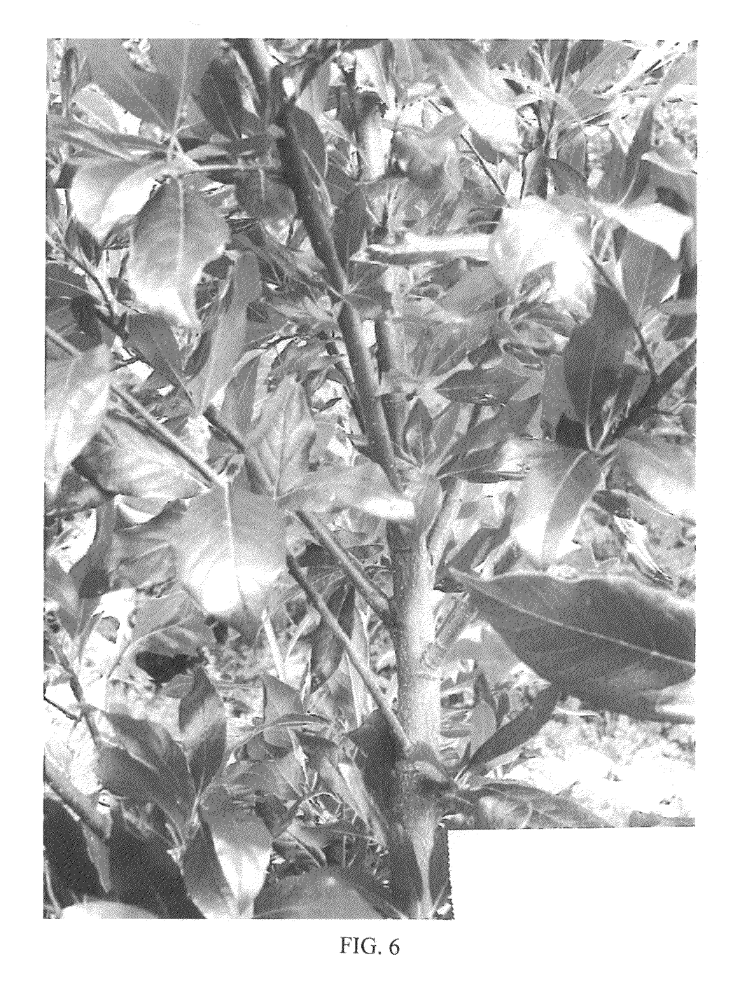

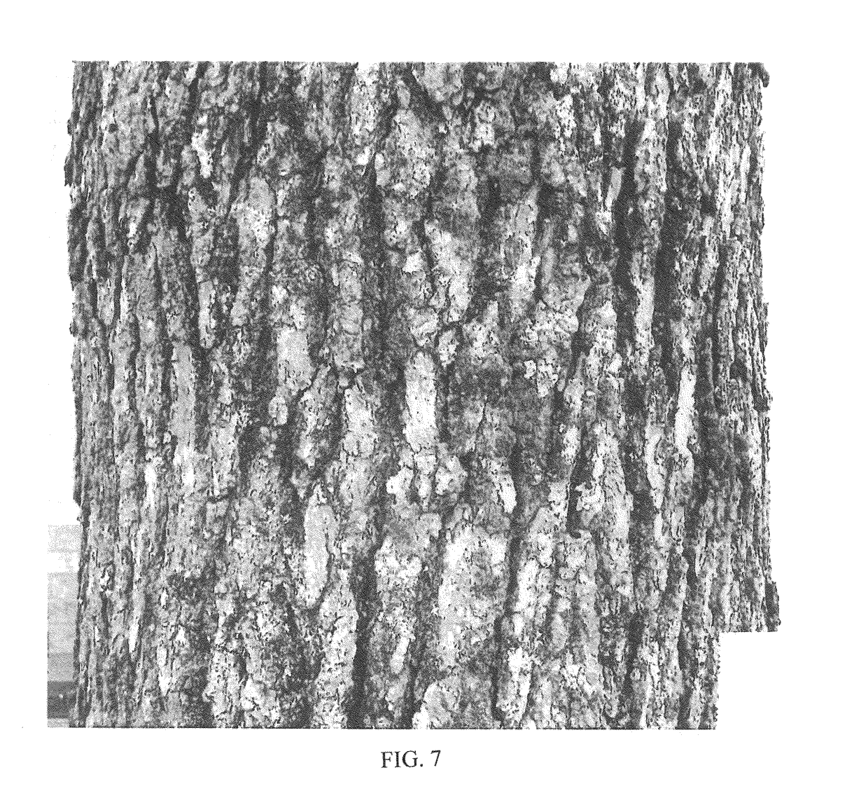

Other than as set below, as of this time no other characteristics have been observed which are different from common Black Gum trees which have been observed by the inventor.
Parentage: Seedling of unknown parentage growing in a cultivated area of a residential landscape. The age of the parent is unknown, but it is approximately 40 feet tall with a spread of 30 feet and a trunk diameter of 20 inches.
Locality where grown and observed: Residential landscape in Murray, Calloway County, Ky.
Leaves: Slightly narrower than the species but otherwise typical: alternate, simple, elliptic, cuneate, acute, smooth, 1 to 1½ inches wide by 4 to 6 inches long; lustrous dark green on the upper surface (RHS 139A) and yellow-green (RHS 145A) on the lower surface (see FIG. 3); fall color of the species varies from fluorescent yellow to orange to red to purple. As shown in FIGS. 1 and 2, leaves of my tree ‘PRP 1’ have a dark crimson red (RHS 46A) fall color.
Petiole: Typical of the species: ½ to 1″ long, green in color.
Buds: Typical of the species: imbricate ⅛ to ¼ inch long, ovoid, reddish in color.
Flowers: Typical of the species: small, greenish yellow flowers borne in 2-4 flowered axillary peduncled clusters. The flowers appear with the leaves and are not ornamentally effective. This species is polygamo-dioecious, with some male trees producing perfect flowers which fruit. My tree appears to be female based on the fruit set.
Fruit: Typical of the species: an oblong drupe, ⅜ to ½ inch long, ripening to blue-black by August through November. The fruit is a good food source for small mammals and birds. Some of the progeny of ‘PRP1’ have fruited, and their fruit production is consistent with that of the parent, ripening as early as late August.
Stem: Typical of the species: slender, glabrous, grayish becoming reddish as the leaves senesce and remaining so through the winter.
Trunk: The parent tree has a large trunk that rises through the canopy as it maintains a central leader. Most trees in the species have large trunks that yield to horizontal branching, creating a rounded canopy lacking a central leader. The bark of the species varies from smooth to scaly to irregularly fissured with color ranging from gray to almost black. As shown in FIG. 7, the bark on ‘PRP1’ is not unusual for the species: grayish, shallowly ridged, and scaly; scales flaking away from the tree reveal grayish brown underneath.
Branching: More upright than the species, which tend to have horizontal to pendulous branching, and unique among the cultivars, which have been primarily selected for fall color and uniform, but not necessarily upright canopies. Branches emerge from the trunk of ‘PRP1’ at an acute angle of 30 to 45 degrees. This is as shown in FIG. 6.
Root system: ‘PRP1’ is grafted onto seedling rootstocks, so the root system is not known. I expect it is typical of the species, which is somewhat coarse.
Growth habit: Pyramidal as a young tree, which is typical, but with upright branching and developing a central leader without diligent pruning and staking, which is an advantage for a grower. The parent tree has a central leader extending to the full height of its canopy, which is an upright oval. This is as shown in FIG. 1.
Vigor: Typical of the species: slow to moderate in its native habitat but vigorous in production.
Diseases: Nyssa sylvatica can be affected by cankers, leaf spots, and rust. Summer leaf spotting is a particular problem which affects most trees, although it is usually only cosmetic. I have observed ‘PRP1’ for several years in the landscape and nursery, and it is relatively free of spotting compared to the species and other cultivars. A typical leaf of ‘PRP1’ is shown in FIG. 3.
Pests: Scale and leaf miners are minor problems which seldom affect the species. I have observed no pest problems with the parent of ‘PRP1’ or its progeny.

1. A new and distinct variety of Black Gum tree named ‘PRP 1’ substantially as herein shown and described, and characterized particularly as to novelty by its well-branched, upright growth habit with a strong central leader, shiny dark green leaves in summer, and a crimson red fall leaf color.
